(19) United States
(12) Patent Application Publication
(10) Pub. No.: US 2004/0167805 A1
(43) Pub. Date: Aug. 26, 2004

(54) Medical pack

(76) Inventors: Adam S. Landsman, Chicago, IL (US); Jason R. Hanft, Coral Gables, FL (US)

Correspondence Address: Michael B. McMurry, 1210 Astor Street., Chicago, IL, 60610, US

(21) Appl. No.: 10/781,424

(22) Filed: Feb. 18, 2004

Related U.S. Application Data
(60) Provisional application No. 60/448,275, filed on Feb. 19, 2003.

(51) Int. Cl. A61B 19/00

(52) U.S. Cl. 705/3; 128/898

(57) ABSTRACT

In order to improve outcomes for a variety of medical conditions that involve home treatment, medical packs are provided that can include a set number of medications and other medical supplies the amounts of which that are selected for a specific period of home treatment. The medical packs can also include instructions using photographs and a patient log. Return of the pack in a return visit to the treating medical professional can provide a means for evaluating patient compliance.

### FIELD OF THE INVENTION

The invention relates to medical packs and in particular to medical packs for home use by patients.

### BACKGROUND OF THE INVENTION

In the past, medical professionals treating various conditions were required to select and assemble materials to give to their patients. This would typically occur in an unorganized and inconsistent fashion. Additionally, with limited experiences to rely on, product recommendations may be less than optimal, and at the same time, much more costly. Disease specific medical kits have been created for improving doctor patient efficiency and helping ensure patient compliance simply by having the correct medicine inside the kit. Examples of medical packs are shown in U.S. Pat. Nos. 5,979,658; 6,382,205 B1; 5,941,241; 5,830,490; and 5,383,891.

However, the medical packs used in the past lack a comprehensive system that helps identify the problem, instruct the patient, dispense the materials, and provide the care all in one package. Also lacking in such medical packs is a effective system of ensuring compliance. Compliance can not only pertain to taking the correct medication but also to the amount of medication taken. Tracking the amount taken can be a difficult task for the physician since he is usually not present when the patient takes the medication. Also, it is advantageous to have varying amounts of medication and medical supplies in medical packs because the same ailment in different people might require different amounts of medication and supplies for treatment. Hence, having medical packs with identical medications and medical supplies but in more specified amounts could help doctors from giving patients more than they need. A comprehensive system of medical packs can reduce costs to both the doctor and the patient.

### SUMMARY OF THE INVENTION

It is therefore an object of the invention to provide a method of assembling a variety of products in a pack or a kit for the home treatment of a specific disease or problem by patients themselves.

Another object of the invention is to provide a pack containing a set number of products necessary to satisfy the requirements of a patient between office visits. For example, if a person has a wound that requires a multi-component bandage, the package would contain the correct amount and mix of products to supply that patient between visits. The pack can also contain printed instructions for usage, including pictures or other images when necessary, in order to assure that the items are being used correctly, and to save the medical professional time by freeing him from having to instruct the patient and answer questions.

A further object of the invention is to provide a pack having a series of products that are optimal for a specific purpose, including mixing products from different manufacturers when necessary, in a single, easy to use and dispense package. As an added advantage, it could free the medical professional from having to write multiple prescriptions, stocking large quantities of items, and doing the research to determine which products are optimal.

Still another object of the invention is to provide a mechanism for marketing, through the collection of data from patients using the products. This also can create a mechanism for ongoing product evaluation and continual improvement of all phases of product development including quality of products, clarity of instructions, and insurance coverage for cost of products.

An additional object of the invention is to provide in each medical pack direction for the medical professional for billing for the products provided. This can include specialized labeling, with adhesive strips containing insurance billing codes.

Yet another object of the invention is to provide a method for categorization of products to allow the medical professional to custom tailor a treatment regimen, in order to improve the level of care rendered.

A further object of the invention is to equip medical packs with starter kits to help the medical professional in selecting the optimum package for a particular disease state.

### BRIEF DESCRIPTION OF THE DRAWING

FIG. 1 illustrates an identifying label that can be applied to the outer portion of a medical pack according to the invention;

FIG. 2 illustrates an inventory table that can be applied to the outer surface of the package or placed inside the package according to the invention for a diabetic wound medical pack;

FIG. 3 depicts a sample doctor instruction sheet that can be placed inside the package;

FIG. 4 depicts a patient log sheet that can be placed inside the package;

FIG. 5 provides a flow chart of relating an ailment to a medical pack; and

FIG. 6 provides a flow chart of patient treatment with the medical pack.

### DETAILED DESCRIPTION OF THE INVENTION

Below is provided a more detailed description of the invention including various methods and criteria for assembling a medical pack along with various uses of such packs and several examples of preferred embodiments of various aspects of the invention.

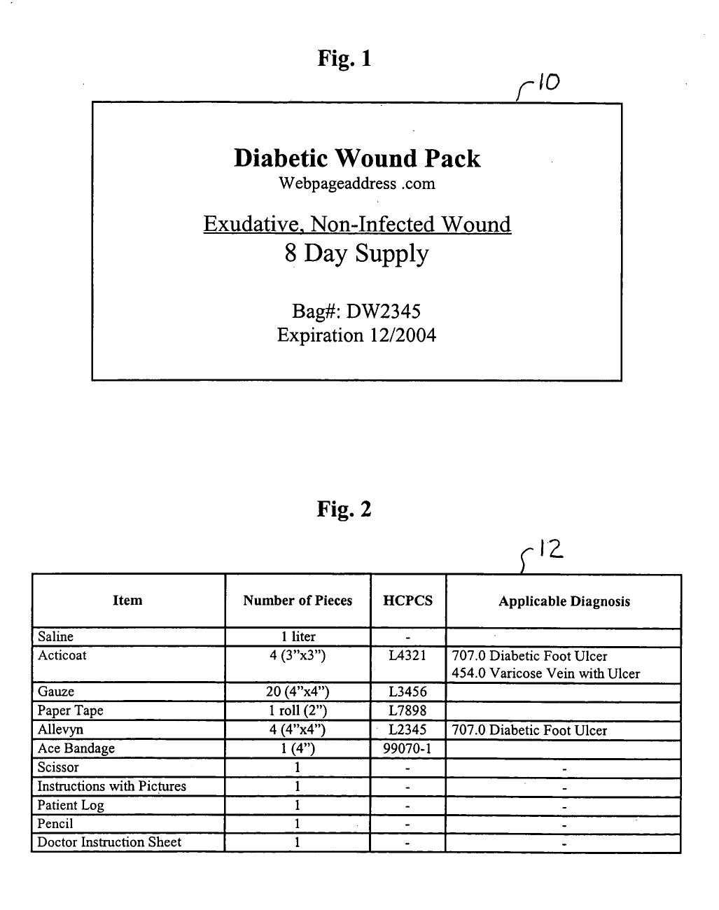

The invention can include a number of different types of medical packs where products in the packs are packaged according to the number of items that will be needed for a specified time period. Preferably, as depicted in FIG. 1, an outer label 10 is applied to an outer surface of the packaging identifying the specific medical pack and the medical condition it is designed to treat along with the duration of the home treatment.

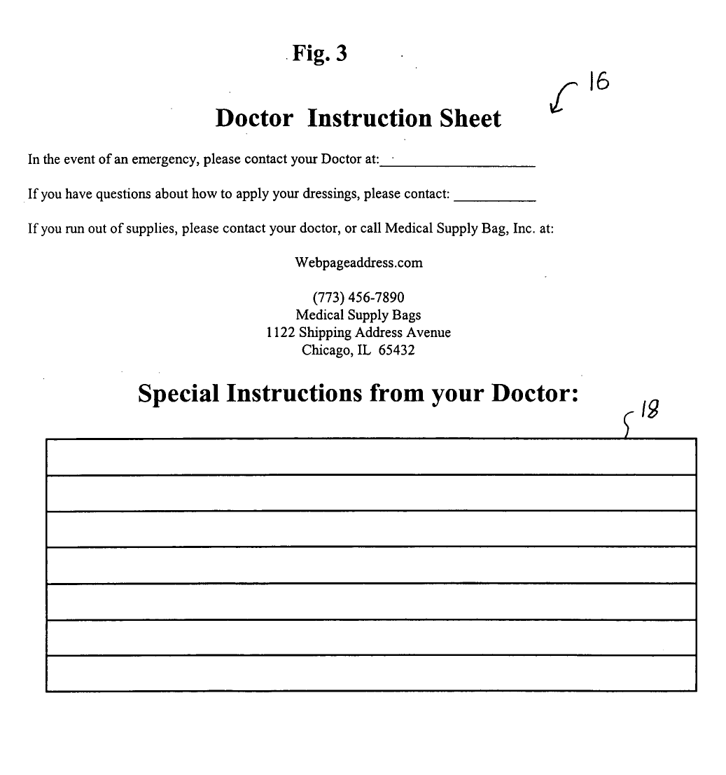

As represented in FIG. 2, an inventory sheet 12 can be applied to the outer surface of the medical pack or alternatively be placed inside the pack. In this example, the inventory sheet 12 lists 20 portions of gauze, 4 Allevyn, and one roll of tape to be used between visits to the doctor. This approach allows the medical professional to monitor usage of the contents of the package. For example, if a patient returns with the pack, and only 15 out of the 20 bandages remain, then it is safe to assume that the patient has not followed directions, and has not been compliant. This packaging can save costs by not requiring the patient to purchase large quantities of items, which are not needed. Also, the packaging can save the patient steps by having all integrated parts together in one simple to use package.

Another aspect of the invention is the provision of a set of instructions for use of all items. Preferably, the instructions include written instructions along with diagrams or a set of photos 14. An example of such a sheet containing photos is contained in U.S. Provisional Patent Application Serial No. 60/448,275 which is incorporated herein by reference. Packaging with this type of instruction system aides in ensuring that the products are used properly, thereby enhancing the patient outcome through increased compliance. Detailed instructions can free the medical professional from having to go through the necessary steps that are required to train the patient when using the products. Detailed instructions can also help ensure that the medical pack is used in the same manner each time, thereby reducing variability when evaluating their usefulness. Instructions can be available in a variety of languages, facilitating communication between patients and medical professionals that may not communicate in the same language. When necessary, instructions can in addition be made available in a variety of formats including CD-ROM, DVD, paper, videotape and cassettes.

Products found in the package will preferably be optimized to work together and to control costs. The mixture of specific items selected can be based on the product's individual and collective performance, as evaluated by a variety of sources, including scientific literature, clinical experience, and patient/consumer feedback. In some cases it might be desirable to provide products that are unique to the medical pack, including those manufactured specifically for the kits, and those which come from companies that have exclusive agreements with the kit providers. In some embodiments, the mixture of products can be provided by several manufacturers. This approach can save the medical professional time by not requiring multiple prescriptions or instructions. As a result, the treatment process is simplified because the process identifies the specific ailment, instructs the patient without the doctor, dispenses the medical supplies, and provides the necessary care all in one package.

This package can be convenient for the patient, who can simply pick it up from his medical professional. It can also save the medical professional time by not requiring him to write multiple prescriptions for the patient to fill, and not requiring the medical professional to stock numerous products. Reorders are convenient since all materials can be delivered under a single product code. Labels clearly indicate the disease state and sub-category that the package is designed for. For example, under the heading of “diabetic foot ulcer,” there can be separate packs for infected wounds, exudative wounds, and deep wounds. The outer labels 10 and 12 show an inventory list and expiration dates as seen in FIGS. 1 and 2.

The outer label 12 of the package can also contain payment codes such as HCPCS codes. Use of codes such as HCPCS codes can be derived from the manufacturer of individual products, and/or can also be studied and suggested by the medical pack provider. Simple pull-off stickers make it easy for medical professionals and coders to submit charges for supplies dispensed. Along with HCPCS codes, estimated reimbursement levels can also be available on the package if desired. Disease state management codes can also be used to increase revenue to the medical professionals.

Preferably, the packages will contain items that have been pre-selected for a particular condition or disease state. In addition to providing a convenience to both patients and doctors, it also serves the purpose of guiding treatment. In the preferred method, the medical professional identifies the condition and selects the correct package. The contents of the pack are used in a self-guided manner, and will help the patient to achieve optimal outcome, even with minimal direction from the medical professional. Doctors who are able to make the proper diagnosis, but have less knowledge about the proper treatment regimen can prescribe a pack and ask the patient to follow the directions, without going in to the specifics of the treatment. A variety of highly focused and broad use packages can be used in the targeted care of a variety of conditions. Also, direct patient involvement in disease management generally improves outcomes.

In addition, products tailored to specific conditions and disease states can provide several avenues for marketing. Critical demographic data can be retrieved from users via mail, internet, etc., which will help to ascertain the quality and outcome of the package and its contents. This demographic and health oriented data can serve as a research tool by monitoring outcomes with the package products. Targeted marketing can be achieved by collecting demographic data from pack users, and distributing packs with samples and/or coupons and directed advertisements for people with a specific condition or disease state. Mailing lists, user outcomes, and a host of other easily retrieved data may be used to enhance medical pack development, or offer guidelines to product manufacturers.

In addition to the various products and advertising materials that can be included in the package, a sheet 16, as shown in FIG. 3, containing a set of additional instructions 18 from the medical professional and directions to home health care providers can be included in the medical pack.

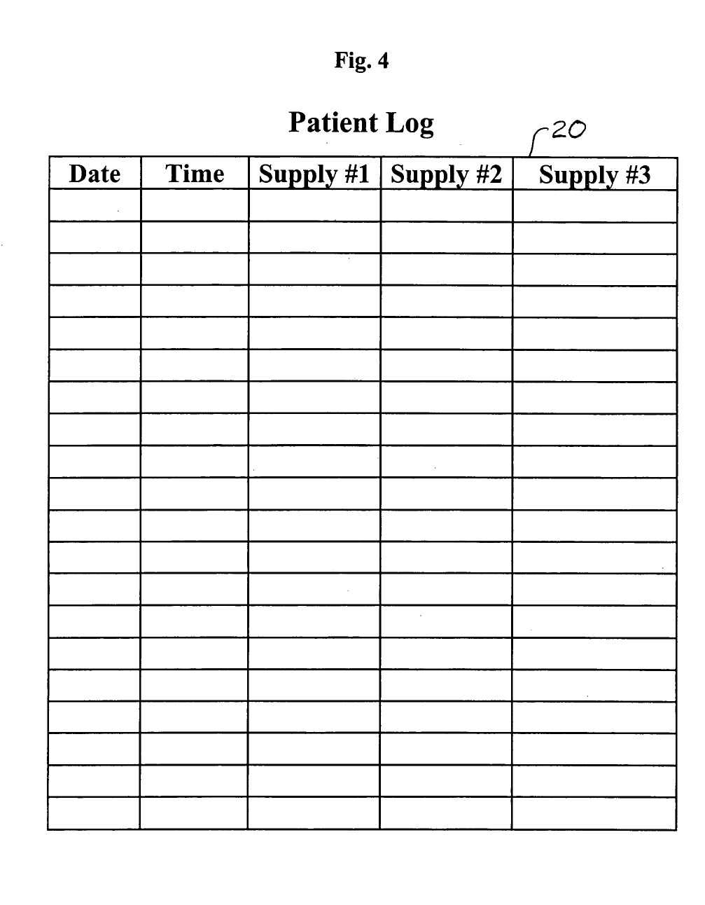

A check-off type log 20, shown in FIG. 4, can also be included so that the patient can indicate the date and time when products were used. Preferably, the patient log should be made as simple as possible. The log 20 can be very useful in permitting medical professionals to gauge compliance and to further customize the usage of packs, as necessary.

Table 1 lists representative examples of disease states for which medical packs can be assembled:

TABLE 1

| | | Pressure | Post-op wounds- | Ostomy/Stoma |
|---|---|---|---|---|
| Diabetic Wounds | Venous Wounds | Wounds | Belly | System |
| Acute laceration or | Arthritis- | Osteoporosis | Post-menopause | Non Insulin |
| wound | Rheumatoid, | | female/hormone | Dependent |
| | Osteoarthritis | | therapy | Diabetes |
| Insulin Depenent | Hypertension/CAD | Asthma | Cataract/Eye Pack | Dialysis/Chronic |
| Diabetes | | | | Renal Failure |
| Skin | Hair Loss(male & | Sexual | Podiatry- Ingrown | Burns |
| Cancer/Melanoma/ | female) | Dysfunction | Nails, Fungus | |
| Dermatologic | | | Nail/Skin, Post-op | |
| | | | bunion/toe, Heel | |
| | | | Pain | |
| Gerneral Cold & F1 | Facial/Plastic | Pregnancy | Maternity/Delivery | Cold |
| Kid's Pack (Travel | Surgery Pack | Pack | Day Pack | Weather/Frostbite |
| Pack) | | | | Pack |

In addition to packs fabricated for patients, educational and equipment packs assembled for use by the medical professional can also be produced. An example of a medical pack and associated materials for treating a diabetic foot wound can include:

Educational Materials for the Doctor

(Diabetic Foot Wound Pack)

Educational Piece (use PPG)

Assess Vascular Supply

Assess Neuologic Function

Assess Wound

Depth and Size

(+) or (−) Infection; Culture and Sensitivity

Skin

Bone—X-ray, MRI

Classification (UTHS)

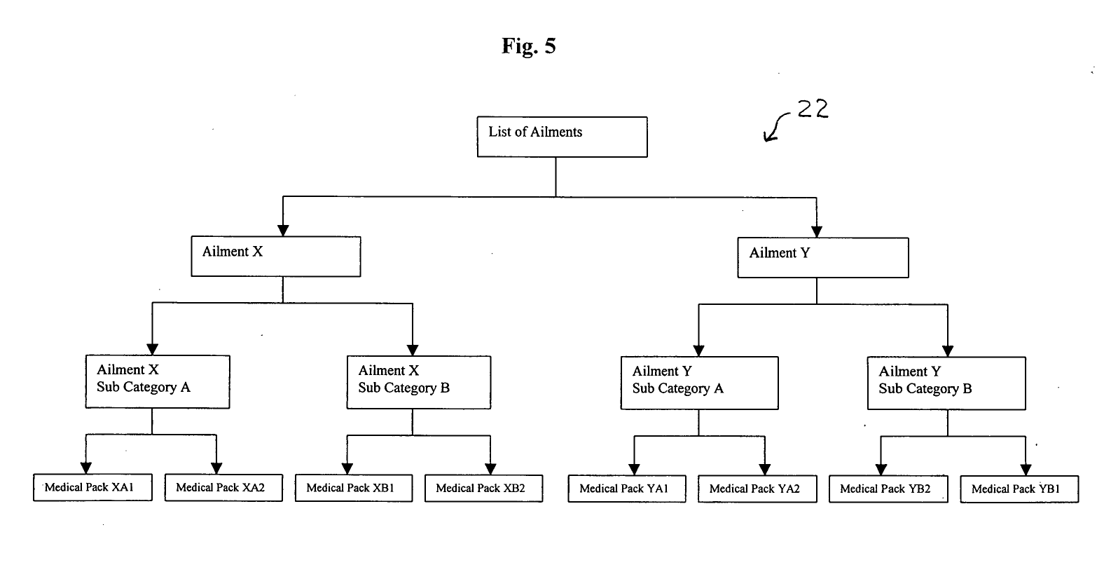

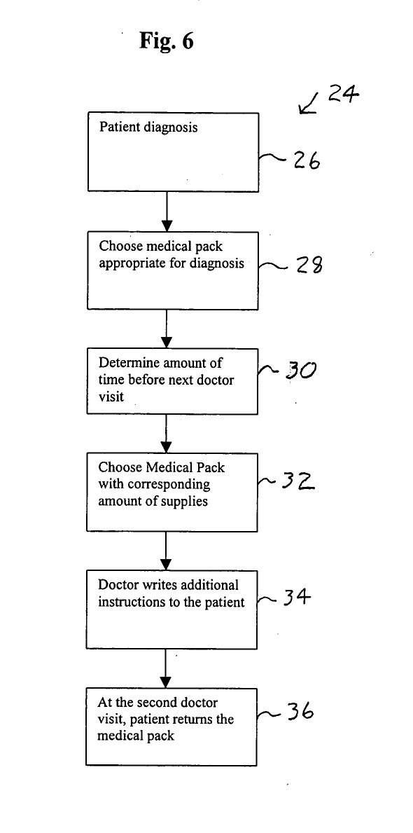

Based on the medical professional's assessment of the wound a specific pack can be selected The election is based on a “Doctor Scorecard” or a flow chart 22, a general example of which is provided in FIG. 5, to point towards a specific pack.

A sample doctor kit can include evaluation tools:

Doctor's Starter Kit—Tools for Evaluation

Complete Kit

Doppler+Cuffs

Sems Weinstein+Tuning Fork+Sharp/Dull

Measuring Device, Sterile Probe

Debridement Tools

Digital Camcorder

User Kit Evaluation (survey for to improve pack contents)

The medical professional can then select a patient medical pack, which can include, for example, the following items:

Patient Medical Pack

Contents: (8 day supply)

(Diabetic Foot Wound)

Daily Kit

Small pack 4×4 gauze

Hydrogel

Topical Antibiotic

Foam Dressing

Tape-Hypofix

Soft Kling Gauze

Saline

Regranex

Dermagraft Option to Add

Panafil to diversify pack

Acticote

Prouramon

PT Instructions

Inspect-Contact doctor ASAP if necessary

(provide contact information)

Limit weightbearing

Wound Care Instructions

Shoe/Brace Instructions

A general description of the packaging materials for this example is as follows:

1. Outer label with adhesive stickers

a. HCPCS Codes and associated linked diagnosis

b. Sticker for chart to indicate which pack was ordered

i. Would include contact address

2. Re-sealable bag with handle

3. Printed instructions

a. instructions may include photos to demonstrate application process as shown in FIGS. 1A-B.

b. check-off boxes to indicate frequency of dressing changes

c. area to write in special instructions

4. Patient Log

a. record daily blood glucose

b. record date/time of dressing changes

5. Pen or Pencil

6. Dressing materials packaged as used

a. Saline

b. Sterile water

c. Tape

d. Other supplies

7. Literature for patient to read (i.e. danger signs when you have a foot ulcer)

8. Targeted advertisement (i.e. web pages, new oral insulin formulations, samples of diabetic candy, order forms for diabetic socks, etc.)

9. Packs targeted to reflect doctor's evaluation of the wound.

a. Clean, non-infected, non-exudative (low bacteria count)

b. Clean, non-infected, exudative

c. Deep wound, with exudate, non-infected

d. Deep wound, with exudates; infected (high bacteria count)

Potential revenue sources resulting from the invention can include: physicians who purchase the medical packs to treat patients, patients, advertising, placement of product samples in packs and sale of database information.

FIG. 6 is a flow chart 24 that describes an example of how the medical pack can be used in treatment. In a first step 26, the ailment of a patient is diagnosed by a doctor. In a second step 28, the doctor chooses a specific pack with the help of the flow chart 22 of FIG. 5, or another device provided in the doctor startup kit. In a next step 30, the doctor determines the amount of time until the next patient visit. The doctor then moves on to a next step 32 and selects a medical kit with the appropriate amount of supplies. The doctor can then fill out the additional instructions 18 of FIG. 3 at a step 34, if he feels it necessary. In a final step 36, the patient returns with the medical kit and the log 20 for the next visit and the doctor can inspect the medical kit to see if the patient complied with his instructions.

In this example of the process 24, the medical pack provides a simple and convenient method of dispensing a variety of well integrated products for treatment of a specific disease state or condition. It allows greater time efficiency, for both the medical professional and patient, by providing detailed, pre-packaged instructions.

Also it allows greater cost efficiency, by providing the user and medical professional with appropriate codes to be used to pursue insurance funding. Additional cost benefits are achieved by eliminating waste through the limiting of quantities of materials distributed to patients. Manufacturer participation will likely lower supply costs.

The medical pack concept described above also offers continuous feedback to the medical professional through the use of patient use logs and continuous feedback to the manufacturers through the use of feedback devices. The medical packs also provide a method to implement tailored care plans devised by experts in each specific field.

We claim:

1. A method for the treatment of patient medical conditions comprising the steps of:
diagnosing the specific medical condition to be treated by a physician;
providing by said physician to the patient a medical pack having a predetermined amount of medical supplies and medication wherein said predetermined amount is sufficient to treat said medical condition for a predetermined time period;
treating said medical condition by said patient for said predetermined time period; and
returning said medical pack to said physician by said patient after said time period.

2. The method of claim 1 wherein said medical packs contain written or photographic instructions for said patient on how to treat said ailment at home.

3. The method of claim 1 wherein said medical packs contain a sheet for additional instructions from said physician to said patient.

4. The method of claim 1 wherein said medical pack contains a log sheet for the patient to record said medical supply and said medication use and date of use.

5. The method of claim 1 wherein said medical pack is evaluated by the doctor and patient.

6. A method of producing specialized medical kits comprising the steps of:
identifying a plurality of medical conditions;
specifying a course of home patient treatment having a predetermined time period for each of said medical conditions;
assembling and packaging a plurality of medical packs each having a predetermined amount of medical supplies and medications for treating said medical conditions for said predetermined time periods.

7. The method of claim 6 wherein a product code is applied to said packaging of said medical packs.

8. The method of claim 6 wherein at least one insurance or billing code is added to said packaging of said medical packs.

9. The method of claim 6 wherein said medical packs integrate said medical supplies and said medication from different manufacturers.

10. A method for the home treatment of a predetermined number of medical conditions comprising the steps of:
creating a medical pack for patient home use that is specific to each said medical condition; and
providing each said medical pack with a predetermined amount of medical supplies and medication for treating each category and subcategory of said medical condition for a predetermined time period;

11. The method of claim 10 wherein at least some of said medical packs contain a starter kit for a physician to use in conjunction with said medical kits.

12. The method of claim 10 including a chart for relating said medical condition to the corresponding medical pack.

13. The method of claim 12 wherein said chart is used by said physician to locate the appropriate medical pack for the patient.
